(19) United States
(12) Patent Application Publication
(10) Pub. No.: US 2010/0121237 A1
(43) Pub. Date: May 13, 2010

(54) ARM SLING AND DECORATIVE SLING JACKET

(76) Inventors: Linda S. Ylisela, Homer Glen, IL (US); Sharon L. Christian, Orland Park, IL (US)

Correspondence Address: Husch Blackwell Sanders, LLP;Husch Blackwell Sanders LLP Welsh & Katz, 120 S RIVERSIDE PLAZA, 22ND FLOOR, CHICAGO, IL, 60606, US

(21) Appl. No.: 12/604,736

(22) Filed: Oct. 23, 2009

Related U.S. Application Data
(60) Provisional application No. 61/197,208, filed on Oct. 24, 2008.

(51) Int. Cl. A61F 5/37

(52) U.S. Cl. 602/4

(57) ABSTRACT

The invention comprises an arm sling comprising a trough formed by front and back sections, each section having a pair of longitudinal generally parallel edges, the front and back sections being secured at the longitudinal edges on an upper side by fastening means, the arm sling having a first open end for placement proximate a hand and a second end for placement about an arm, the first open end having a thumb loop secured thereto for engagement with the thumb of a user; a pocket attached to the sling for the placement inside the sling, a neck strap connected to opposite ends of said arm sling, the neck strap having hooks and loops on opening end portions for adjusting the strap to a desired length.

### FIELD OF THE INVENTION

The present invention concerns an arm sling for use by persons having an arm injury requiring the immobilization of an arm or portion thereof. More particularly, the present invention relates to an arm sling and decorative jacket that allows an injured person to comply with medical requests for immobilization of the limb while remaining comfortable and stylish.

### BACKGROUND OF THE INVENTION

Arm slings are used for retaining/protecting/supporting the arm of a patient following an injury or illness. A variety of arm slings currently available on the market consist of a fabric support into which the patient's forearm is placed with the fingers or hand partially or fully extending from the front end of the pouch. These slings further have a strap attached to the support in such a manner that the support can be used to hold the arm within the pouch in a desired position as required for treatment. Typically the strap is affixed near the elbow end of the sling, is drawn over the shoulder of the injured arm and is attached to the front end of the pouch, through a variety of attachment means such as a large buckle.

We have found that as these existing slings must be made of durable materials, to provide necessary support and ease in care and cleaning of the sling, they are relatively uncomfortable against the skin of the user. Materials typically used in slings, include but are not limited to, polyester, nylon, and webbing. Further, we have found that pouches of present day slings are not custom made for the users and are often quite large, to accommodate a variety of sizes of users, allowing more extensive movement of the arm within than may be advisable in most injury situations.

While these slings have a long history of being fully capable of supporting an arm and provide some small degree of protection to the injured arm, they are however bulky, uncomfortable and have a utilitarian look. Further they are usually offered as one-size-fits-all in a world where individual size and tastes are now catered. As a result it would be desirable to have a means to support an arm in a manner that is medically necessary while being comfortable both in the manner that the arm is held and in tactile ways. All of this could be accomplished while allowing the wearer to represent their own sense of taste in what is, for all purposes, a garment, while immobilization of the arm is maintained. It would also be desirable to have an arm support that could be easily changed or covered to provide protection of the arm in inclement weather or to provide a different look in accordance with the taste of the user.

Accordingly, there is a need for an arm sling that adequately stabilizes an individual's arm and upper shoulder and additionally provides as much comfort as possible during use of the sling. Thus, an orthopedic arm sling solving the aforementioned problems is desired.

### SUMMARY OF THE INVENTION

The orthopedic arm sling is a sling designed to stabilize the upper shoulder and arm of a user. The orthopedic arm sling includes a folded sheet of fabric material defining a sling trough into which an arm is placed and a plurality of straps that allow the arm of the user to be supported within the trough and suspended from the user's neck.

In accordance with the present invention, there is provided comfort, support, stability, adjustability and style. The fabrics offered are attractive and comfortable, and may be in a variety of colors. In addition, noting that many patients now are conscious of irritating or allergic-causing materials, the arm sling is made of 100% organic cotton material.

The invention may be in six sizes, XS, S, M, L, XL and L. The length of the arm is measured from the wrist to the elbow to determine the proper size. Sizing is key to proper fit; from the thumb loop location, the length of the arm, the fit of the elbow and the fit of the upper arm.

The present invention is made from one piece of fabric, sewn together from the elbow to the upper arm, providing a pouch with two identical front and back sections. There is no bottom seam to irritate the patient's arm and all seams are covered in seam binding for additional protection. The strap is sewn into a deep seam in the back middle section of the upper arm. The patient's forearm is placed in the pouch and the strap is drawn over the shoulder, through a single loop buckle. The strap is adjusted for proper length in the positioning of the arm.

The upper arm/bicep section is adjustable via a strip of hook and loop material that is sewn to the upper arm section, and attaches to itself by mating hook and loop material sewn at the end of the strip. This allows the patient to properly fit the upper arm of the sling to their size thereby providing a more tailored and secure fit.

The compatible snap system is sewn to the front and back forearm sections and encloses the invention over the top of the forearm, offering added protection and support.

To prevent the arm from sliding forward out of the sling, a thumb loop is strategically placed and sewn to the front and back sections at the end of the sling, providing additional security for the patient.

Thus, the invention comprises an arm sling comprising a trough formed by front and back sections, each section having a pair of longitudinal generally parallel edges, the front and back sections being secured at the longitudinal edges on an upper side by fastening means, the arm sling having a first open end for placement proximate a hand and a second end for placement about an arm, the first open end having a thumb loop secured thereto for engagement with the thumb of a user; a pocket attached to the sling for the placement inside the sling, a neck strap connected to opposite ends of said arm sling, the neck strap having hooks and loops on opening end portions for adjusting the strap to a desired length.

Thus, the invention may further include an arm sling comprising a trough, the trough fitting over the arm from the wrist to the bicep of the arm, the sling having a cover, the cover being secured to the sling by a hook and loop section on the cover secured to a mating hook and loop section on the sling.

A more detailed explanation of the invention is provided in the following description and claims and is illustrated in the accompanying drawings.

### BRIEF DESCRIPTION OF THE DRAWINGS

FIG. 1 is a side view of the arm sling of the subject invention.

FIG. 2 is a side view of a second embodiment of the arm sling of the subject invention.

FIG. 3 is a front view of the arm sling of the subject invention supporting an arm of a wearer.

FIG. 4 is a top view showing a jacket for use with the arm sling of the subject invention.

FIG. 5 is a front view of a user with a jacket covering the arm sling of the subject invention.

DETAILED DESCRIPTION OF THE ILLUSTRATIVE EMBODIMENT

While the present invention is susceptible of embodiment in various forms, there is shown in the drawings a number of presently preferred embodiments that are discussed in greater detail hereafter. It should be understood that the present disclosure is to be considered as an exemplification of the present invention, and is not intended to limit the invention to the specific embodiments illustrated. It should be further understood that the title of this section of this application (“Detailed Description of an Illustrative Embodiment”) relates to a requirement of the United States Patent Office, and should not be found to limit the subject matter disclosed herein.

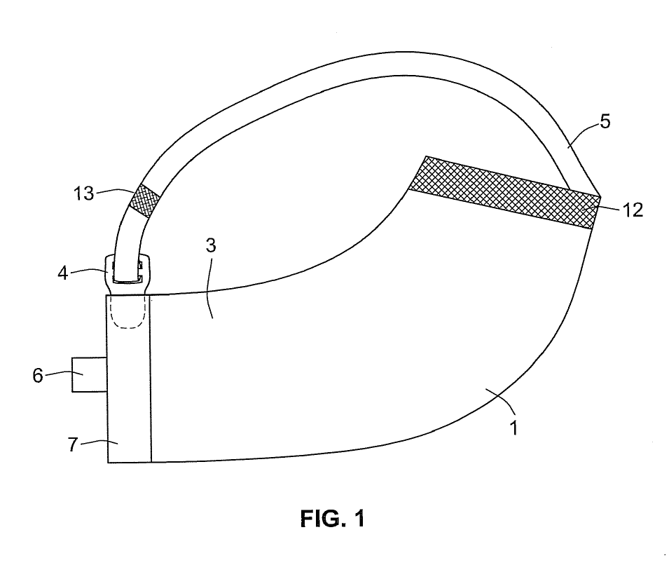

As shown in FIG. 1, in sling 15 both front and back sections are identical, providing equal stability and support for the patient's arm/shoulder/wrist. The strap 5 is sewn into the deep seam in the back middle section where the two front and back sections are sewn together to form a trough. The seam is reinforced for extra strength. The free end of the strap 5 draws over the patient's shoulder and passed through the single loop buckle 4, overlapping itself and attaching to the strap by hook material 13. This strap 5 can be adjusted by use of hook and loop strips at various lengths depending on the length requirements of the patient.

A strip of hook and loop material 2 is sewn onto the upper arm/bicep section of the arm sling. A thumb loop 6 is sewn to the front and back sections 1 at the outer end of the sling placed so as to permit the thumb to be easily grasped by the loop and thus prevent the arm from forward out of the sling.

The fabric 3 used for the sling in an embodiment, is 100% organic cotton, stretch cotton, French terry or stretch cotton twill in a variety of colors. The high quality fabrics maintain an even weight distribution of the arm, help to relieve weight on the neck muscles and are breathable, therefore providing more airflow through the arm sling. The fabrics should be soft to the skin of the patient, eliminating that “itchy” feeling.

A cuff 7 may be located at the outer end of the sling. The cuffed design provides fashion, avoiding an industrial or utilitarian appearance.

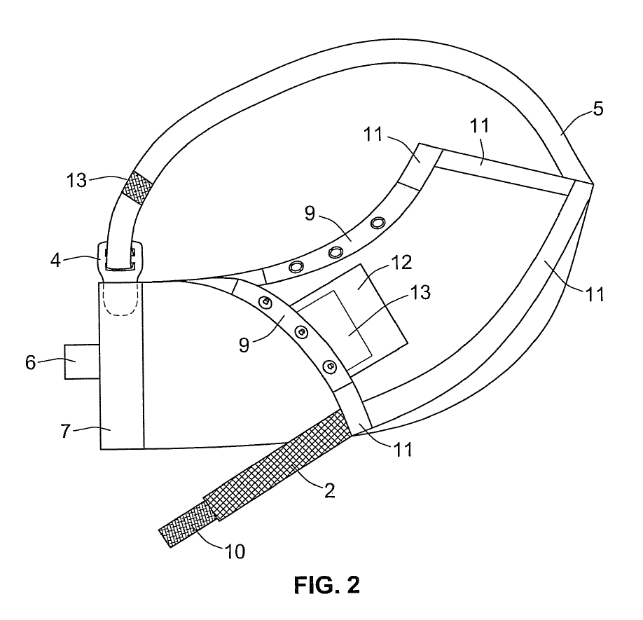

As shown in FIG. 2, a series of snaps 9 are sewn into the front and back forearm sections, and upon the patient placing their arm in the pouch, the snaps may be secured, allowing the sling front and back sections to cover the forearm. A soft loops and hooks compatible strip of material 2, is sewn onto the upper arm/bicep section of the sling. The loose end of the strip 2 can then attach to itself at the proper size adjustment by a sewn piece of hook material 13.

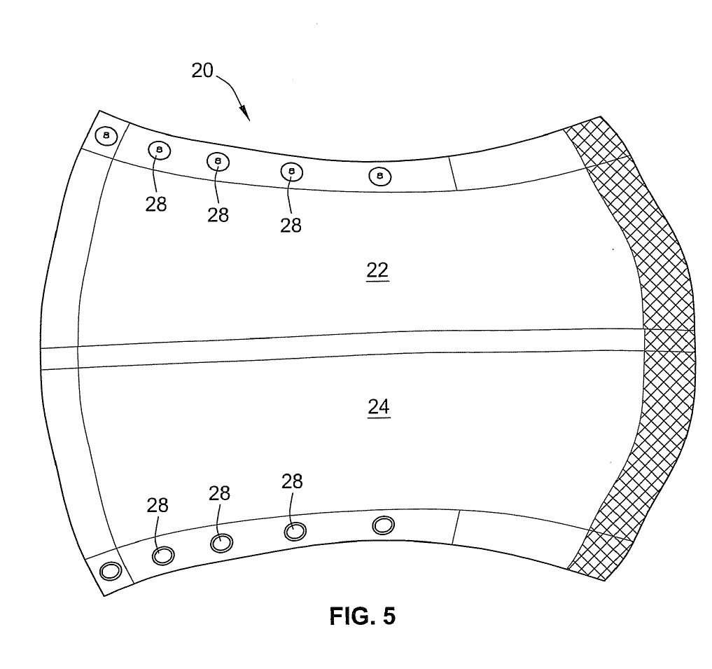

All raw edges inside the sling and jacket 20 (FIG. 5) may be covered with seam binding 11, to help alleviate any skin irritation to the patient.

An inside pocket 12 for holding and securing small valuables, keys and the like, may be attached to the sling for placement inside the sling, such as by being sewn into the seam of the sling; the pocket has a flap 14, that folds over the pocket to help prevent items from falling out of the pocket.

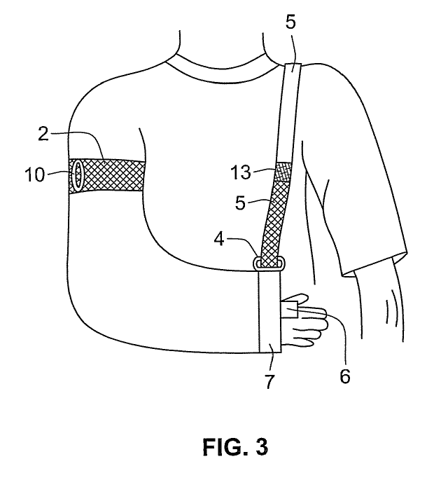

As shown in FIG. 3, the upper arm is adjustable by a hook and loop strip 2 that is secured onto the upper arm/bicep section of the sling. The strip self attaches via hook and loop material 10, to a mating section on the sling to adjust to the proper size for the patient. The strap 5 is drawn over the shoulder of the patient and passes through the single loop buckle 4, then self-attaches via mating hook and loop material 13 at the end of the strap. The patient's thumb may be drawn through the thumb loop 6, which is sewn to both front and back sections of the sling, preventing the patient's arm from sliding forward and out of the sling.

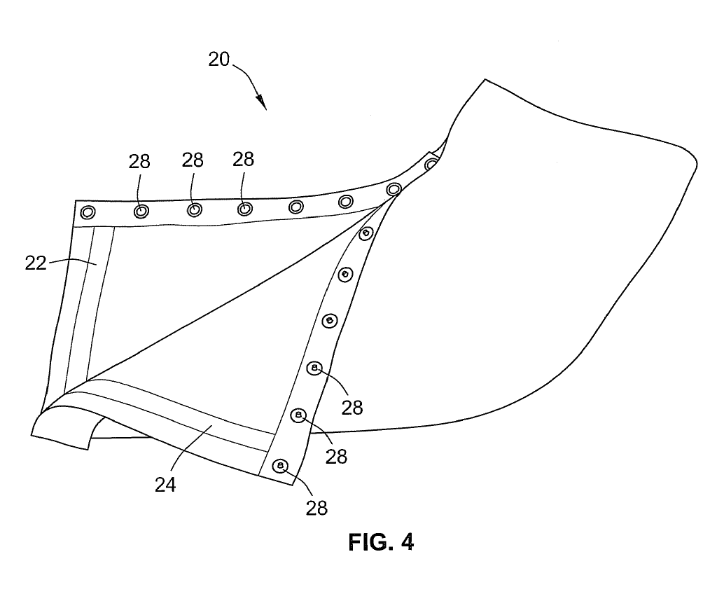

As shown in FIGS. 4 and 5, a jacket 20 may be used to cover the arm sling of the subject invention. Thus, the jacket may provide a wide range of options related to fabric choices, graphic designs and colors for the patient wearing the arm sling. The jacket has two panel sections 22 and 24 that match the corresponding sections of the sling, and completely covers the sling.

The inside front and back forearm sections are lined with a mating compatible snap system 28, providing the ability to snap close over the front and back forearm sections of the sling. The jacket may be secured to the sling by a hook and loop portion 25 on the jacket that mates with a corresponding hook and loop portion on the sling.

The sling jacket is available in sizes that are compatible with the sizes of the sling, and therefore are form fitting.

Although an illustrative embodiment of the invention has been shown and described, it is to be understood that various modifications and substitutions may be made by those skilled in the art without departing from the novel spirit and scope of the invention.

What is claimed is:

1. An arm sling comprising a trough formed by front and back sections, each section having a pair of longitudinal generally parallel edges, said front and back sections being attached at the longitudinal edges on an upper side by fastening means, said arm sling having a first open end for placement proximate a hand and a second end for placement about an arm, said first opening having a thumb loop secured thereto for engagement with the thumb of a user; a pocket attached to the sling for placement inside the sling, a neck strap connected to opposite ends of said arm sling, said neck strap having hooks and loops on opposing end portions for engagement with mating sections on said arm sling to adjust the strap to a desired length.

2. The arm sling of claim 1 wherein the fastening means are snaps.

3. The arm sling of claim 1 wherein the thumb loop engages the thumb to prevent the arm from sliding out of the sling.

4. The arm sling of claim 1 wherein said pocket has a flap.

5. The arm sling of claim 1 wherein the sling is supported about a user by a strap, said strap being adjustable in length.

6. The arm sling of claim 5 wherein the strap has a section of hooks and loops for attaching to a mating section on said sling.

7. The arm sling of claim 1 further including a cuff on the first open end.

8. An arm sling comprising a trough, said trough fitting over the arm from the wrist to the bicep of the arm, said sling having a cover, said cover being secured to said sling by a hook and loop section on said cover secured to a mating hook and loop section on said sling, said cover being selected from a plurality of covers, each said cover having a different graphic design.

9. The arm sling of claim 9, wherein the hook and loop section on said cover is on an inside portion of said cover, and hook and loop section on said sling is on an outside portion of said sling.
